(19) United States
(12) Patent Application Publication
(10) Pub. No.: US 2014/0166019 A1
(43) Pub. Date: Jun. 19, 2014

(54) HEADGEAR APPARATUS FOR NASAL INTERFACE

(71) Applicants: Peter Chi Fai Ho, PittSburgh, PA (US); Jerome Matula, JR., Apollo, PA (US)

(72) Inventors: Peter Chi Fai Ho, PittSburgh, PA (US); Jerome Matula, JR., Apollo, PA (US)

(73) Assignee: KONINKLIJKE PHILIPS N.V., EINDHOVEN (NL)

(21) Appl. No.: 14/232,060

(22) PCT Filed: Jul. 19, 2012
(86) PCT No.: PCT/IB2012/053684

§ 371 Date: Jan. 10, 2014

Related U.S. Application Data
(60) Provisional application No. 61/511,611, filed on Jul. 26, 2011.

(51) Int. Cl. A61M 16/06

(52) U.S. Cl. CPC A61M 16/0683; A61M 16/0666
USPC 128/207.13; 128/206.21

(57) ABSTRACT

An improved headgear is structured to secure a mask to the head of a patient and comprises a parietal support, an occipital support, and a zygomatic support connected together. The zygomatic support comprises a pair of zygomatic braces that are each structured to engage the face of the patient and to support the mask in fluid communication with the patient. The zygomatic braces each comprise at least one of a cephalic element structured to engage the face cephalic to the crest of the zygomatic bone, and a caudal element structured to engage the face caudal to the zygomatic bone.

### CROSS-REFERENCE TO RELATED APPLICATIONS

This patent application claims the priority benefit under 35 U.S.C. §119(e) of U.S. Provisional Application No. 61/511,611 filed on Jul. 26, 2011, the contents of which are herein incorporated by reference.

### BACKGROUND OF THE INVENTION

1. Field of the Invention

The present invention pertains to respiratory interface devices and, in particular, to headgear for use in securing a mask to a patient.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder such as sleep apnea syndrome in particular, obstructive sleep apnea (OSA), or congestive heart failure.

Non-invasive ventilation and pressure support therapies involve the placement of a respiratory patient interface device including a mask component on the face of a patient. The mask component may be, without limitation, a nasal mask that covers the patient's nose, a nasal cushion having nasal prongs that are received within the patient's nares, a nasal/oral mask that covers the nose and mouth, or full face mask that covers the patient's face. The respiratory patient interface device interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head. Because such respiratory patient interface devices are typically worn for an extended period of time, it is important for the headgear to maintain the mask component of the device in a tight enough seal against the patient's face without discomfort.

For respiratory patient interface devices, a key engineering challenge is to balance patient comfort against stability of the device. As a patient changes sleeping positions through the course of the night, the mask portions of respiratory patient interface devices may become dislodged, and the seal against the patient may be broken. A dislodged mask portion can be stabilized by the increasing strapping force provided by the headgear, but increased strapping force tends to reduce patient comfort. This design conflict is further complicated by the widely varying facial geometries that a given respiratory patient interface device design needs to accommodate.

### SUMMARY OF THE INVENTION

Accordingly, it is an object of the present invention to provide a headgear for use in securing a respiratory patient interface to the head of a patient that overcomes the shortcomings of conventional headgear.

This object is achieved according to at least one embodiment of the present invention by providing an improved headgear for use in securing a nasal pillow interface to the head of a patient. The headgear can be characterized as including parietal support, an occipital support, and a zygomatic support that together form the headgear and which is used to secure the nasal pillow interface to the patient. In a number of the embodiments of the headgear, the zygomatic support includes a zygomatic brace that is configured to be situated across the zygomatic bone or caudal to the zygomatic bone of the patient in order to maintain desirable positioning of the headgear and the nasal pillow interface. As employed herein, the expression “a number of and variations thereof shall refer broadly to any non-zero quantity, including a quantity of one. As employed herein, the expressions “cheekbone”, “zygomatic bone”, “zygomatic arch”, and “zygomatic crest” refer broadly and generally to any one or more of a variety of facial features such as the zygomatic bone, the zygomatic process of the temporal bone, the temporal process of the zygomatic bone, and/or the soft tissue or other structures in the zygomatic region of a patient.

In a number of embodiments the zygomatic support includes a number of stiffening structures that enhance the rigidity of the zygomatic support in certain planes while still maintaining flexibility in other planes. Additionally or alternatively, the parietal support or the occipital support or both can include such stiffening structures. Moreover, it is expressly noted that any of the various features of the various depicted embodiments can be combined in any fashion with any of the features of other embodiments in any combination to result in additional embodiments that are not expressly depicted herein but are nevertheless considered to be within the scope of the disclosure.

In certain of the embodiments depicted herein, the headgear is formed at least partially of a rigid but flexible silicone polymer that is molded to have a specific three-dimensional shape when in a free state. The headgear in other embodiments depicted herein is formed of a flexible silicone rubber or other synthetic material and has the shape of a flat strap or a nonspecific shape when in a free state.

Certain of the embodiments depicted herein additionally employ a conventional fabric strap as the parietal support or the occipital support.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

In certain embodiments, the general nature of the invention can be stated as including a headgear structured for use in securing a mask to the head of a patient and including a parietal support, at least a portion of which is structured to extend across a parietal region of the head; an occipital support, at least a portion of which is structured to extend across an occipital region of the head; and a zygomatic support, at least a portion of which is structured to extend across a zygomatic region of the head. The parietal, occipital, and zygomatic supports are connected together, and the zygomatic support includes a pair of zygomatic braces that are each structured to engage the face of the patient and to support the mask in fluid communication with the patient. The zygomatic braces each include at least one of a cephalic element structured to engage the face of the patient in a region cephalic to the crest of the zygomatic bone, and a caudal element being structured to engage the face of the patient in a region caudal to the zygomatic bone.

In certain embodiments, the general nature of the invention could be stated as including a headgear apparatus that includes a nasal interface mask structured to be in fluid communication with the head of a patient and a headgear structured to support the nasal interface mask in fluid communication with the head of the patient. The headgear can be stated as including a parietal support, at least a portion of which is structured to extend across a parietal region of the head; an occipital support, at least a portion of which is structured to extend across an occipital region of the head; and a zygomatic support, at least a portion of which is structured to extend across a zygomatic region of the head. The parietal, occipital, and zygomatic supports are connected together, and the zygomatic support includes a pair of zygomatic braces that are each structured to engage the face of the patient and to support the mask in fluid communication with the patient. The zygomatic braces each include at least one of a cephalic element structured to engage the face of the patient in a region cephalic to the crest of the zygomatic bone, and a caudal element being structured to engage the face of the patient in a region caudal to the zygomatic bone.

### BRIEF DESCRIPTION OF THE DRAWINGS

FIG. 1 is a perspective view of an improved headgear in accordance with a first embodiment of the invention situated on a patient and positioning a nasal pillow interface on the patient;

FIG. 2 is a front elevational view of the headgear of FIG. 1 and the nasal pillow interface situated on the patient;

FIG. 3 is an enlarged front view of a portion of a zygomatic strap at the left of the headgear FIG. 2;

FIG. 4 is an enlarged view of the underside of a portion of a zygomatic strap at the right of the headgear FIG. 2;

FIG. 5 is a sectional view as taken along line 5-5 of FIG. 4;

FIG. 6 is an enlarged front elevational view of a portion of an alternative zygomatic strap of an alternative zygomatic support of a second embodiment of a headgear in accordance with the present invention;

FIG. 7 is an enlarged view of the underside of a zygomatic brace of the alternative zygomatic strap depicted generally in FIG. 6;

FIG. 8 is a sectional view as taken along line 8-8 of FIG. 7;

FIG. 9 is a side elevational view of an improved headgear in accordance with a third embodiment of the present invention situated on a patient and positioning a nasal pillow interface on the patient;

FIG. 10 is an enlarged sectional view as taken along line 10-10 of FIG. 9;

FIG. 11 is a perspective view of an improved headgear in accordance with a fourth embodiment of the present invention situated on a patient and positioning a nasal pillow interface on the patient;

FIG. 12 is an enlarged sectional view as taken along line 12-12 of FIG. 11;

FIG. 13 is a side elevational view of an improved headgear in accordance with a fifth embodiment of the present invention situated on a patient and positioning a nasal pillow interface on the patient;

FIG. 14 is an enlarged sectional view as taken along line 14-14 of FIG. 13;

FIG. 15 is an enlarged sectional view as taken along line 15-15 of FIG. 13;

FIG. 16 is an enlarged sectional view as taken along line 16-16 of FIG. 13;

FIG. 17 is a side elevational view of an improved headgear in accordance with a sixth embodiment of the present invention situated on a patient and positioning a nasal pillow interface on the patient;

FIG. 18 is an enlarged sectional view as taken along line 18-18 of FIG. 17;

FIG. 19 is an enlarged sectional view as taken along line 19-19 of FIG. 17;

FIG. 20 is an enlarged sectional view as taken along line 20-20 of FIG. 17;

FIG. 21 is an enlarged sectional view as taken along line 21-21 of FIG. 17;

FIG. 22 is an enlarged sectional view as taken along line 22-22 of FIG. 17;

FIG. 23 is an enlarged sectional view as taken along line 23-23 of FIG. 17;

FIG. 24 is a side elevational view of an improved headgear in accordance with a seventh embodiment of the present invention situated on a patient and positioning a nasal pillow interface on the patient;

FIG. 25 is an enlarged sectional view as taken along line 25-25 of FIG. 24;

FIG. 26 is an enlarged sectional view as taken along line 26-26 of FIG. 24;

FIG. 27 is a side elevational view of an improved headgear in accordance with an eighth embodiment of the present invention situated on a patient and positioning a nasal pillow interface on the patient;

FIG. 28 is a side elevational view of an improved headgear in accordance with a ninth embodiment of the present invention situated on a patient and positioning a nasal pillow interface on the patient;

FIG. 29 is an enlarged view of a portion of a zygomatic support of the headgear of FIG. 28;

FIG. 30 is an enlarged sectional view as taken along line 30-30 of FIG. 29;

FIG. 31 is an enlarged view of a portion of an alternative zygomatic strap that can be employed in a tenth embodiment of the improved headgear;

FIG. 32 is an enlarged view of a portion of another alternative zygomatic strap that can be employed in a eleventh embodiment of the improved headgear; and

FIG. 33 is an enlarged view of a portion of another alternative zygomatic strap that can be employed in a twelfth embodiment of the improved headgear.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

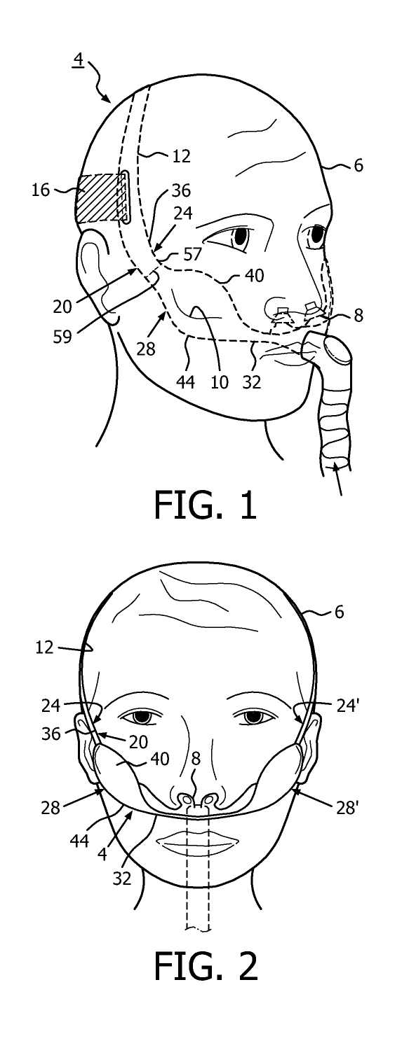

An improved headgear 4 in accordance with a first embodiment of the present invention is depicted in FIG. 1 as being situated on a patient 6 and as supporting a nasal pillow interface 8 engaged with the nostrils of patient 6. As is generally understood, patient 6 has a zygomatic bone or cheekbone that is depicted schematically at the numeral 10 and which is a facial bone. Headgear 4 advantageously interacts with zygomatic bone 10 in order to support and maintain the position of headgear 4 and nasal pillow interface 8 with respect to patient 6. More particularly, at least a portion of headgear 4 interacts with the soft tissue of the face of patient 6 in the region of zygomatic bone 10. Headgear 4 and nasal pillow interface 8 can together be referred to as a headgear apparatus.

While the headgear apparatus employs nasal pillow interface 8 to provide fluid communication with the breathing passages of patient 6, it is understood that nasal pillow interface 8 is merely an example of one such patient interface. Other types of interfaces of any type, such as other nasal interfaces, nasal cushions, oral interfaces, or combinations of nasal and oral interfaces may be employed in conjunction with headgear 4 or in conjunction with any other embodiments thereof that are described or otherwise contemplated herein, without limitation.

Headgear 4 can be characterized as including a parietal support 12, an occipital support 16, and a zygomatic support 20 that are connected together. Parietal support 12 can be said to be situated approximately along the parietal bone of patient 6, and occipital support 16 can be generally said to lie approximately across the occipital bone of patient 6, it being noted that the parietal and occipital bones are known cranial bones. While zygomatic bone 10 and the parietal and occipital bones (not expressly depicted herein) are mentioned herein in connection with headgear 4, it is noted that such facial and cranial bones are intended merely for purposes of illustration and could be referred to generally by the expressions “zygomatic region”, “parietal region”, and “occipital region” without departing from the present disclosure.

Occipital support 16 is depicted in FIG. 1 as being a conventional fabric or other known flexible strap. Parietal support 12 and zygomatic support 20, however, are depicted as being co-formed as a unitary single piece member having a specific three-dimensional shape when in a free state. The combined occipital and zygomatic supports 16 and 20 are formed of a rigid but flexible silicone polymer material that provides a certain level of rigidity to maintain the position of zygomatic support 20 with respect to zygomatic bone 10, i.e., overlying zygomatic bone 10, while still providing a level of flexibility to enable it to conform to the facial structure of patient 6.

Zygomatic support 20 can be said to include a zygomatic strap 24 and a zygomatic brace 28, with zygomatic strap 24 including an anterior portion 32 extending from zygomatic brace 28 generally toward nasal pillow interface 8 and as further including a posterior portion 36 extending generally from zygomatic brace 28 toward parietal support 12. As can be understood from FIG. 1, zygomatic brace 28 overlies zygomatic bone 10, whereby zygomatic bone 10 provides positional support to zygomatic brace 28 and thus to headgear 4.

Zygomatic strap 24 and zygomatic brace 28 are depicted at the left side of FIG. 2, and it is noted that zygomatic support 20 of headgear 4 actually further includes another zygomatic strap 24′ and another zygomatic brace 28′ which are depicted at the right side of FIG. 2. The two zygomatic straps 24 and 24′ and the two zygomatic braces 28 and 28′ are mirror images of one another. Other headgear embodiments will be set forth below, and while typically only one side of each such headgear embodiment is depicted and described for purposes of simplicity, it is expressly noted that the other side of each such headgear embodiment is a mirror image of the depicted and described side.

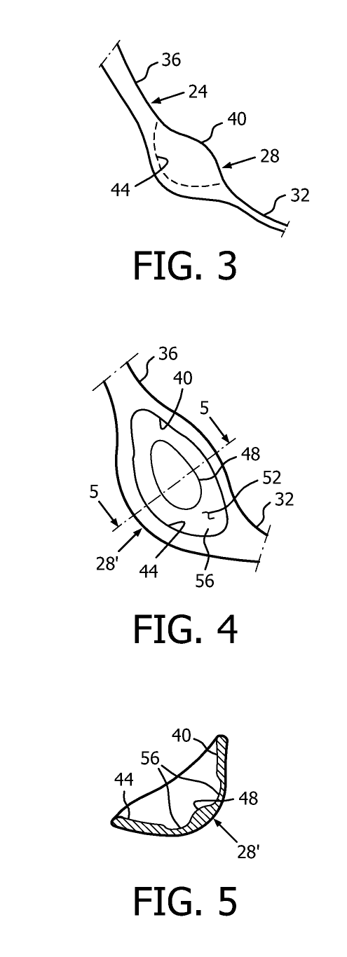

FIGS. 3-5 depict zygomatic brace 28 as being generally of a cup shape and including a cephalic element 40, a caudal element 44, and an intermediate element 48 that is situated generally between cephalic and caudal elements 40 and 44. Zygomatic brace 28 can be said to further include a region of reduced thickness 52 which forms a linking element 56 that extends between and connects intermediate element 48 with cephalic and caudal elements 40 and 44. More particularly, the thicknesses of cephalic element 40, caudal element 44, and intermediate element 48 are depicted in FIG. 5 as being crosshatched, as is the thickness of linking element 56, but it can be seen from FIG. 5 that linking element 56 (which is formed by region of reduced thickness 52) is of a relatively thinner cross-section than cephalic, caudal, and intermediate elements 40, 44, and 48. As such, linking element 56 is relatively more flexible for movements in the plane of the page of FIG. 5 than any of cephalic, caudal, and intermediate elements 40, 44, and 48.

Thus, when zygomatic brace 28 is engaged with zygomatic bone 10, it is noted that cephalic, caudal, and intermediate elements 40, 44, and 48 will maintain substantially their shape depicted generally in FIG. 5 whereas linking element 56 will deform to allow zygomatic brace 28 to follow the shape of zygomatic bone 10 in order to accommodate the various shapes of the various facial bones of the various patients in connection with which headgear 4 will be used. Linking element 56 extends about the entire perimeter of intermediate element 48 and thus isolates intermediate element 56 from cephalic and caudal elements 40 and 44.

It is understood that in other embodiments of headgear 4 that are not expressly depicted herein, at least a portion of region of reduced thickness 52 could be in the form of a region of zero thickness, i.e., a region where no material exists, such as would result from an opening formed in zygomatic brace 28. In a situation where only a portion of region of reduced thickness 52 is a region of zero thickness, linking element 56 would have some type of opening formed therein. Alternatively, in a situation where the entirety of region of reduced thickness 52 is a region of zero thickness, linking element 56 would not exist in zygomatic brace 28, and rather zygomatic brace 28 would have an opening in the place of linking element 56.

As can be generally understood from FIGS. 1-5, cephalic element 40 generally overlies a portion of zygomatic bone 10 that is cephalic to, i.e., above, the crest of zygomatic bone 10, it being understood that the crest of zygomatic bone 10 is schematically indicated in FIG. 1 to represent zygomatic bone 10. Caudal element 44 can be said to be in the form of a wall that is situated generally caudal to, i.e., underneath, zygomatic bone 10. Intermediate element 48 can be said to generally engage the crest of zygomatic bone 10.

As has been set forth elsewhere herein, headgear 4 and, more particularly, the combined parietal and zygomatic supports 12 and 20 that are co-formed, possess a specific shape in three dimensions when in a free state, and it is thus understood that when headgear 4 is mounted on patient 6, zygomatic brace 28 will deform slightly and advantageously to conform with the particular shape of the particular zygomatic bone 10 of the particular patient 6. In this regard, linking element 56 formed by region of reduced thickness 52 will deform, i.e., bend, to a significantly greater extent than any of cephalic, caudal, and intermediate elements 40, 44, and 48, thus enabling the geometry of caudal element 44, for instance, to maintain its shape while being advantageously positioned in a region caudal to zygomatic bone 10.

Similarly, the geometry of cephalic element 40 remains generally unchanged, but deformation of linking element 56 enables cephalic element 40 to overlie a region of zygomatic bone 10 that is cephalic to the crest of zygomatic bone 10. Linking element 56 provided by region of reduced thickness 52 thus advantageously enables zygomatic brace 28, which has a specific three-dimensional shape when in a free state, to accommodate various facial and cranial shapes of various patients by conforming to and engaging the patient's particular zygomatic bone when headgear 4 is mounted on patient 6.

Moreover, it can be understood from FIG. 1 that posterior portion 36 of zygomatic strap 24 includes a curve element 57 which causes zygomatic strap 24 to be positioned generally anterior to parietal strap 12. As can be understood from FIG. 1, curve element 57 has a transverse dimension in the plane of the page of FIG. 1 which is the thickness of zygomatic strap 24 and which is at a minimum along the axis designated by the numeral 59, and the dimension along the axis 59 that is transverse to the thickness is significantly greater than the thickness of curve element 57, i.e., several times the thickness, at the same location in a direction toward the face of patient 6. As such, curve element 57 and posterior portion 36 are relatively deflectable about axis 59 but are relatively rigid about an axis (not expressly depicted herein) that is oriented orthogonal to axis 59 and into the plane of the page of FIG. 1. That is, the region of smallest transverse dimension as is indicated along axis 59 permits flexing of zygomatic brace 28 in a direction generally toward zygomatic bone 10 in order to enable zygomatic brace 28 to engage the various zygomatic bones of the various patients with which headgear 4 will be used, which is desirable.

Also, some twisting of zygomatic brace 28 along an axis of elongation of zygomatic support 20 will be permitted and will further enable zygomatic brace 28 to engage the various zygomatic bones of the various patients. However, the relatively large transverse dimension of posterior portion 36 along axis 59 when compared with the thickness of posterior portion 36 as can be understood from FIG. 2 limits bending movement of posterior portion 36 about an axis that is positioned orthogonal to axis 59 and is oriented generally into the face of patient 6. As such, curve element 57 is advantageously configured to cause zygomatic brace 28 to be positioned generally overlying zygomatic bone 10, and the relative flexibility of posterior portion 36 about axis 59 advantageously enables zygomatic brace 28 to be flexed and twisted into engagement with zygomatic bone 10 as set forth above, with zygomatic brace 28 itself advantageously deforming, i.e., bending and twisting, along linking element 56 to enable zygomatic brace 28 to further conform to the particular shape of zygomatic bone 10.

It thus can be seen that certain features of headgear 4, such as the configuration of posterior portion 36 at axis 59, enable certain relative flexing of headgear 4, such as in the way posterior portion 36 can relatively bend about axis 59 and twist about the axis of elongation of zygomatic support 20. However, certain features of headgear 4 also resist flexing of headgear 4 in other planes, such as in the way flexing of posterior portion 36 is resisted about an axis orthogonal to axis 59 and into the face of patient 6. Thus, curve element 57 orients zygomatic brace 28 with respect to zygomatic bone 10, and it permits bending of zygomatic brace 28 generally toward zygomatic bone 10 and twisting of zygomatic brace 28 in order to enable engagement with zygomatic bone 10, which advantageously supports headgear 4 on patient 6.

As can be understood from FIG. 4, which generally depicts an underside of zygomatic brace 28′, i.e., the surface of zygomatic brace 28′ that engages the skin of patient 6 at zygomatic bone 10, the region of reduced thickness 52 and the resultant linking element 56 extend fully about intermediate element 48. That is, intermediate element 48 can be said to be isolated from cephalic and caudal elements 40 and 44 by region of reduced thickness 52. As such, linking element 56 enables intermediate element 48 to move, by deformation of linking element 56, in generally all directions with respect to cephalic and caudal elements 40 and 44.

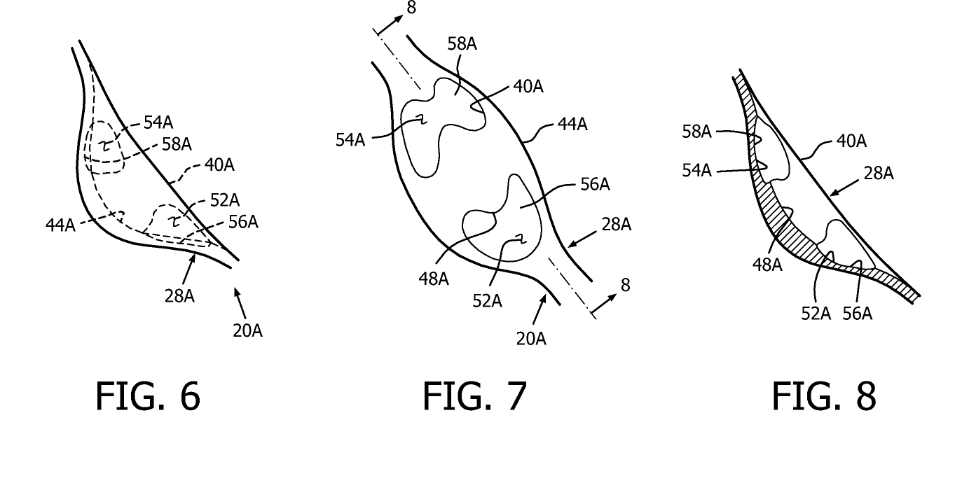

An alternative to zygomatic brace 28 is depicted generally at the numeral 28A in FIGS. 6-8, which depict an alternative zygomatic support 28A which could be incorporated into headgear 4 in place of zygomatic brace 28 and which would result in another improved headgear (not expressly depicted herein) in accordance with a second embodiment of the present invention. It is noted that the second embodiment would be formed by replacing, for instance, in FIG. 1 zygomatic brace 28 with zygomatic brace 28A from FIGS. 6-8, and thus a complete view of the headgear of the second embodiment is not separately depicted herein for purposes of simplicity of disclosure.

Zygomatic brace 28A can be understood from FIGS. 6-8 to include a cephalic element 40A and a caudal element 44A that function in substantially the same way as cephalic and caudal elements 40 and 44. However, it can be seen from FIGS. 6-8 that zygomatic brace 28A includes an intermediate element 48A that is connected with and extends between cephalic and caudal elements 40A and 44A. In this regard, zygomatic brace 28A includes a pair of regions of reduced thickness, i.e., an anterior region of reduced thickness 52A and a posterior region of reduced thickness 54A disposed anterior and posterior of intermediate element 48A, which result in an anterior linking element 56A and a posterior linking element 58A situated anterior and posterior of intermediate element 48A, respectively.

A zygomatic support 20A that includes zygomatic brace 28A can be said to function in substantially the same way as zygomatic support 20, except that the connection of intermediate element 48A with cephalic and caudal elements 40A and 44A has the effect of limiting the ability of intermediate element 48 to be deflected in certain directions with respect to cephalic and caudal elements 40A and 44A. More particularly, the flexibility afforded by anterior and posterior linking elements 56A and 58A limits to a meaningful extent the flexibility of zygomatic brace 28A to flexing generally only in the plane of the page of FIG. 8 and a certain level of twisting about an axis situated along the longitudinal direction of zygomatic support 20A. Such limited flexibility of zygomatic support 20A can be employed in applications or on particular patients where such limited flexibility is necessary or desirable.

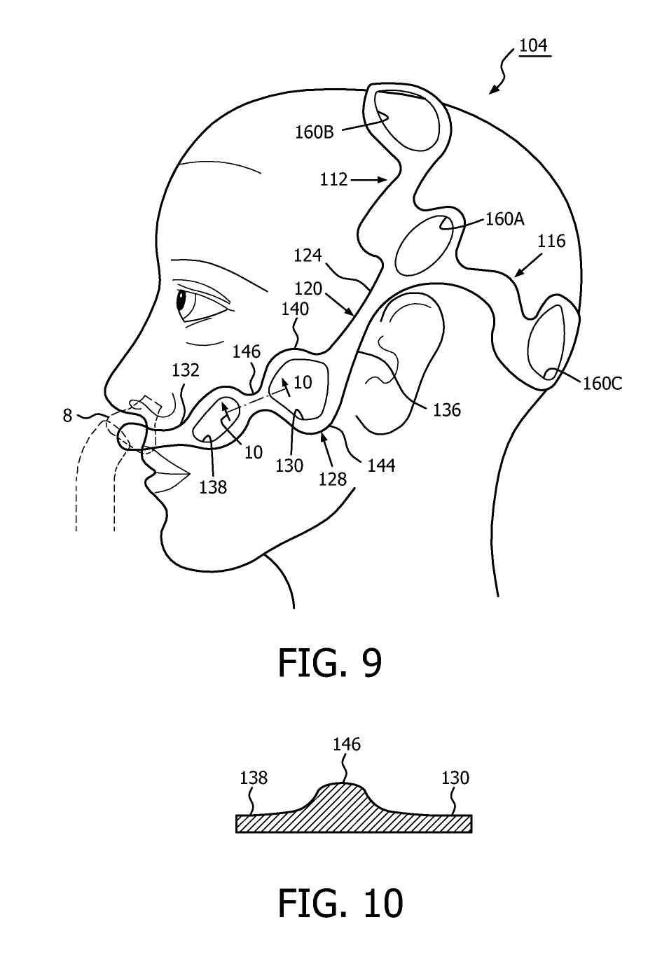

An improved headgear 104 in accordance with a third embodiment of the present invention is depicted generally in FIGS. 9 and 10. Headgear 104 supports a nasal pillow interface 108 on a patient and includes a parietal support 112, an occipital support 116, and a zygomatic support 120. Zygomatic support 120 includes a zygomatic strap 124 that carries a zygomatic brace 128. Notably, zygomatic brace 128 is in the form of a zygomatic loop 130 that interacts with the zygomatic bone of the patient. More particularly, zygomatic loop 130 is situated between an anterior portion 132 of zygomatic strap 124 and a posterior portion 136 of zygomatic strap 124 and includes a cephalic element 140 and a caudal element 144. It can be understood that cephalic element 140 constitutes the cephalic, i.e., upper, segment of zygomatic loop 130, and caudal element 144 constitutes the caudal, i.e., lower, segment of zygomatic loop 130. The cephalic element 144 is situated cephalic to the crest of the zygomatic bone, and caudal element 144 is situated caudal to the crest of the zygomatic bone. By situating portions of zygomatic loop 130 cephalic and caudal to the zygomatic bone, zygomatic loop 130 can be said to at least partially surround at least a portion of the zygomatic bone of the patient, which provides support to zygomatic brace 128 and thus to headgear 104.

Moreover, because headgear 4 is formed of a rigid but flexible silicone polymer material and is of a specific three-dimensional shape when in a relaxed state, zygomatic loop 130 additionally provides directed stiffness to zygomatic support 120. Longitudinal forces directed along posterior portion 136 of zygomatic strap 124 are directed along cephalic and caudal elements 140 and 144 of zygomatic loop 130 to a bridging element 146 of anterior portion 132 to another loop 138 formed in anterior portion 132, and vice versa. That is, forces between posterior portion 136 and anterior portion 132 are directed along zygomatic loop 130, bridging element 146, and the other loop 138. By configuring cephalic and caudal elements 140 and 144 to have particular shapes, the transfer of forces between posterior portion 136 and bridging element 146 can be controlled. Similarly, loop 138 enables stiffening of anterior portion 132 and for the directing of forces between bridging element 146 and the portion of anterior portion 132 that carries nasal pillow interface 108.

It is also noted that bridging element 146 is of a relatively thicker cross-section than the portions of zygomatic loop 130 and the other loop 138 that are connected therewith, as is indicated in FIG. 10. Such increased thickness resists bending at bridging element 146, which further enables accurate directing of forces along the anterior and posterior portions 132 and 136 of zygomatic strap 128, such as those forces that tend to position zygomatic loop 130 on the zygomatic bone of the patient and the forces which reactively provide support to the other portions of headgear 104 with respect to the zygomatic bone.

It is noted that FIG. 9 depicts a number of additional exemplary loops, such as a loop 160A between parietal and occipital supports 112 and 116, a loop 160B in parietal support 112, and a loop 160C in occipital support 116. Such additional loops 160A, 160B, and 160C can be provided as needed to lend stiffness, support, and the directing of forces among various elements of headgear 104, as needed. In this regard, it is understood that the various loops depicted in headgear 104 can be provided at different locations, and the interconnecting components therebetween can likewise be at other positions or in other orientations without departing from the present concept. Moreover, the shapes of the various loops 130, 138, 160A, 160B, and 160C can likewise be different than that expressly depicted herein without departing from the scope of the present invention. Such variation will vary the way in which forces are directed along such loops.

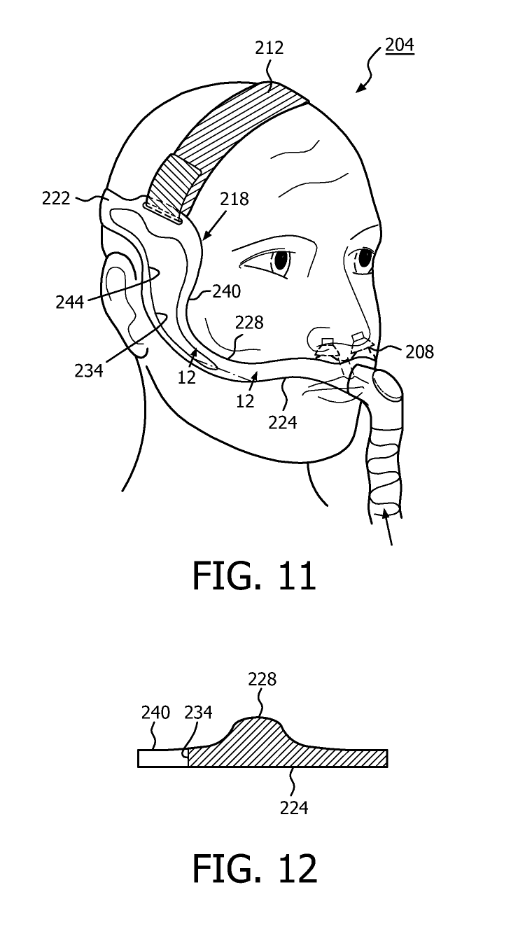

An improved headgear 204 in accordance with a fourth embodiment of the present invention is depicted generally in FIGS. 11 and 12. Headgear 204 is depicted as supporting a nasal pillow interface 208 on a patient. Headgear 204 includes a parietal support 212 that is in the form of a conventional fabric strap that is connected with a zygomatic/occipital support 218 that is co-formed as a single piece member. Zygomatic/occipital support 218 can be said to include an occipital strap 222 and a zygomatic strap 224, with zygomatic strap 224 having a zygomatic brace 228 in the form of thickened region anterior to a zygomatic/occipital loop 234 that spans occipital and zygomatic straps 222 and 224.

Zygomatic/occipital loop 234 directs forces along a cephalic element 240 and a caudal element 244 thereof to provide support for nasal pillow interface 208 while maintaining a desirable position of nasal pillow interface 208 with respect to the zygomatic bone of the patient. More particularly, zygomatic strap 224 is situated caudal to the zygomatic bone of patient and can be said to engage the zygomatic bone at its caudal region. This is possible, because in addition to zygomatic/occipital loop 234 directing forces along its cephalic and caudal elements 240 and 244, zygomatic/occipital loop 234 can stretch along the coronal plane, i.e., vertically with respect to a standing patient, upon tightening of parietal strap 212.

During such stretching, portions of cephalic element 240 are stretched away from portions of caudal element 244. As such, parietal strap 212 can be tightened to the point that zygomatic brace 228 engages the caudal portion of the zygomatic bone, at which point cephalic element 240 will be maintained in a state of equipoise between the tension afforded by parietal support 212 and the engagement of zygomatic brace 228 with the caudal portion of the zygomatic bone. Simultaneously, caudal element 244 will provide a compressive load at zygomatic brace 228, which provides further support to zygomatic strap 224 to resist nasal pillow interface 208 from becoming disengaged with the nostrils of the patient. As can be understood from FIG. 12, zygomatic brace 228 has a relatively thickened cross section in order to promote the directing of forces from cephalic and caudal elements 240 and 244 to zygomatic brace 228 and to zygomatic strap 224 to support nasal pillow interface 208.

As can be best understood from FIG. 11, cephalic element 240 can be said to extend generally across the sphenoid and/or temporal bones of the patient. Caudal element 244 can be said to extend generally across the temporal bone of the patient. It is noted, however, that such positioning of cephalic and caudal elements 240 and 244 is exemplary in nature only, and it is therefore understood that such elements can be positioned to extend across different portions of the cranium and/or face of the patient without departing from the present concept.

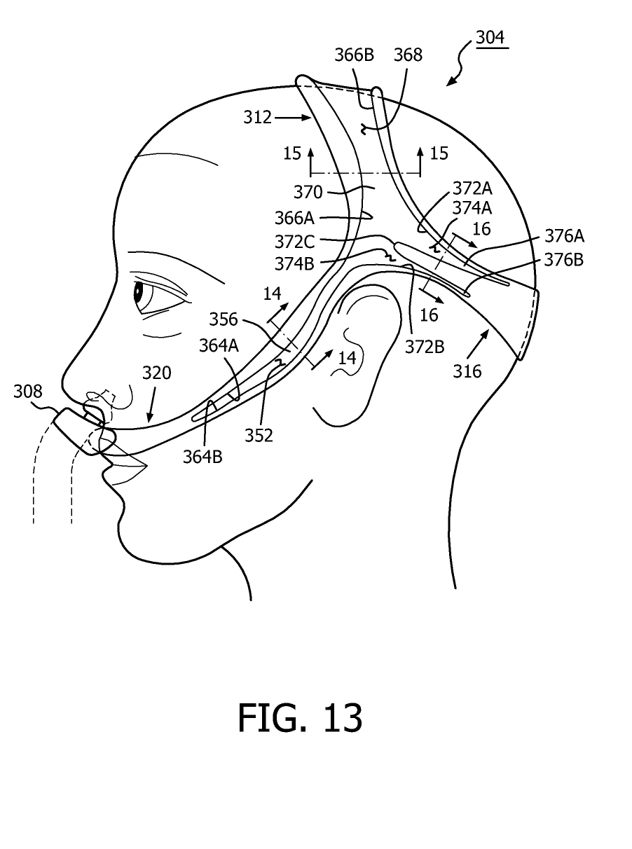

An improved headgear 304 in accordance with a fifth embodiment of the present invention is depicted generally in FIGS. 13-16. Headgear 304 supports a nasal pillow interface 308 in fluid communication with the nostrils of the patient. Headgear 304 includes a parietal support 312, an occipital support 316, and a zygomatic support 320, although in headgear 304 the zygomatic support 320 does not necessarily have any particular interaction with the zygomatic bone of the patient. Rather, and as will be set forth in greater detail below, headgear 304 includes a number of stiffening elements that provide focused rigidity in predetermined fashions while still permitting a desirable degree of flexibility.

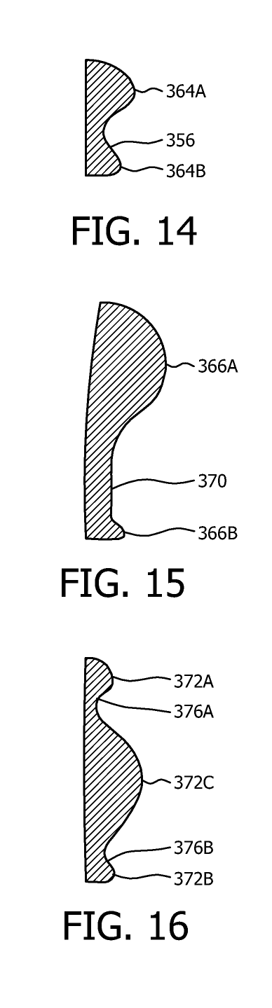

As can be understood from FIGS. 13 and 14, zygomatic support 320 includes an elongated cephalic zygomatic stiffening element 364A and an elongated caudal zygomatic stiffening element 364B that are situated along the upper and lower edges of zygomatic support 320, as is indicated generally at FIG. 14. A zygomatic region of reduced thickness 352 results in a zygomatic linking element 356 situated between cephalic and caudal zygomatic stiffening elements 364A and 364B. As can be understood from FIG. 14, cephalic and caudal zygomatic stiffening elements 364A and 364B are each of a relatively thicker cross section in a direction perpendicular to the skin than zygomatic region of reduced thickness 352. In the exemplary embodiment depicted in FIGS. 13 and 14, cephalic zygomatic stiffening element 364A is thicker than caudal zygomatic stiffening element 364B along the same direction, i.e., perpendicular to the skin of the face.

As can be understood from FIGS. 13 and 15, parietal support 312 includes an anterior parietal stiffening element 366A and a posterior parietal stiffening element 366B that are both elongated and that are of a relatively greater thickness than a parietal region of reduced thickness 368 that forms a parietal linking element 370 between anterior and posterior parietal stiffening elements 366A and 366B.

As can be understood from FIGS. 13 and 16, occipital support 316 includes a cephalic occipital stiffening element 372A, a caudal occipital stiffening element 372B, and an intermediate occipital stiffening element 372C. Intermediate occipital stiffening element 372C is situated between cephalic and caudal occipital stiffening elements 372A and 372B, with the result that occipital support 316 includes both a cephalic occipital region of reduced thickness 374A and a caudal occipital region of reduced thickness 374B. Cephalic occipital region of reduced thickness 374A results in a cephalic occipital linking element 376A situated between a portion of intermediate occipital stiffening element 372C and a portion of cephalic occipital stiffening element 372A. Likewise, caudal occipital region of reduced thickness 374B results in a caudal occipital linking element 376A situated between at least a portion of intermediate occipital stiffening element 372C and caudal occipital stiffening element 372B. As can be understood from FIG. 13, intermediate occipital stiffening element 372C extends generally into the region at which parietal, occipital, and zygomatic supports 312, 316, and 320 connect with one another.

Regardless of the express enumeration of elements set forth above, it can be seen that many of the indicated elements cooperate in one way or another. For instance, cephalic zygomatic stiffening element 364A and anterior parietal stiffening element 366A together form a unitary elongated stiffening element that extends between the zygomatic region and the parietal region. Similarly, caudal zygomatic stiffening element 364B and caudal occipital stiffening element 372B together form a unitary elongated stiffening element that extends between the zygomatic region and the occipital region of the patient. Likewise, posterior parietal stiffening element 366B and cephalic occipital stiffening element 372A together form a unitary elongated stiffening element that extends between the parietal and occipital regions of the patient.

Because headgear 304 has a fixed shape in three dimensions when in a free state, i.e., in a non-installed state, it can be understood that the various stiffening elements depicted in FIGS. 13-16 help to maintain a positional relationship among parietal support 312, occipital support 316, and zygomatic support 320, which maintains nasal pillow interface 308 in fluid communication with the nostrils of the user. Moreover, the various thicknesses and widths of the various stiffening elements and regions of reduced thickness depicted in FIGS. 13-16 enables particularized deflection responses when installed on various patients having various facial and cranial structures. For instance, the relative height of headgear 304 as at each of the indicated section lines 14-14, 15-15, 16-16 affects the stiffness, i.e., resistance to bending, within the plane of the page of FIG. 13.

For instance, if the portion of headgear 304 indicated in FIG. 14 has its height increased in the vertical direction from the perspective of FIG. 14, resistance to bending within the plane of FIG. 13 will be increased. While resistance to bending within another plane (not expressly depicted herein) perpendicular to the plane of the page of FIG. 13 will be increased at least somewhat, its increase will typically not be as much as the increase in resistance to bending within the plane of the page of FIG. 13. As can be understood, by configuring the various stiffening elements and regions of reduced thickness in headgear 304, various stifthesses and flexibilities can be provided that can enable headgear 304 to be accommodated on patients having various facial and cranial structures while still maintaining a desirable level of comfort and maintaining nasal pillow interface 308 in fluid communication with the nostrils of the patient.

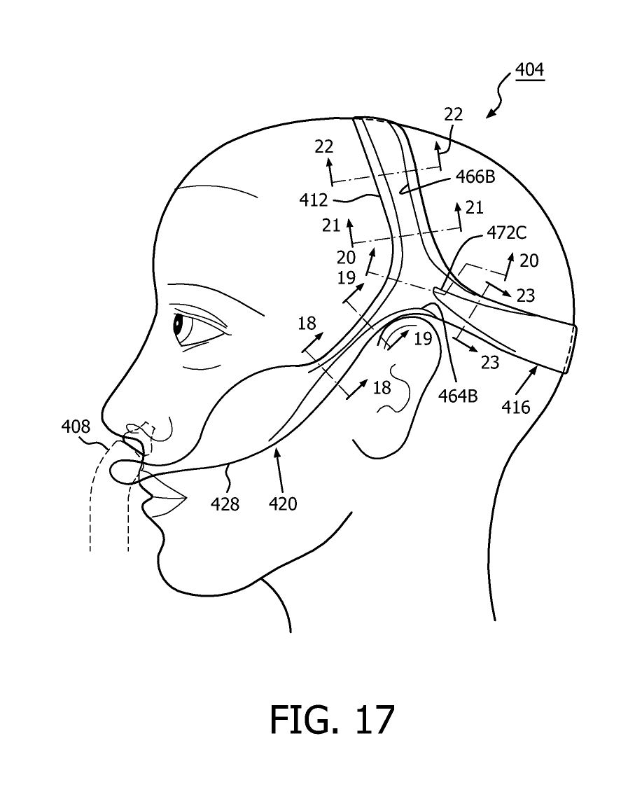

As has been stated elsewhere herein, any of the elements described herein can be combined with any of the other elements described herein to create new combinations of elements that are within the scope of the present disclosure. By way of example, an improved headgear 404 in accordance with a sixth embodiment of the present invention is depicted generally in FIGS. 17-23 and can be said to be a combination of headgear 304 and zygomatic brace 28. That is, headgear 404, which supports a nasal pillow interface 408 in fluid communication with the nostrils of a patient, includes a parietal support 412, an occipital support 416. It further includes a zygomatic support 420 that is similar to zygomatic support 320, except that zygomatic support 420 further includes a zygomatic brace 428 similar to zygomatic brace 28 that interfaces with the zygomatic bone of the patient. The resultant headgear 404 provides an even greater degree of support of nasal pillow interface 408. On the other hand, by providing headgear 404 to include the stiffening elements of headgear 304 in combination with zygomatic brace 428, the various stiffening elements employed in headgear 304 potentially can be made relatively smaller or otherwise provided with a greater degree of flexibility when used in conjunction with zygomatic brace 428 since zygomatic brace 428 itself provides support to headgear 404 by engaging the zygomatic bone region of the patient. By making the various stiffening elements relatively smaller and thus more flexible, greater comfort is achieved for the patient.

By way of example, zygomatic support 420 includes a caudal zygomatic stiffening element 464B that extends generally only to about the region where zygomatic support 420 and occipital support 416 join one another. In contrast, caudal zygomatic stiffening element 364B in FIG. 13 extends along occipital support 316 and meets intermediate occipital stiffening element 372C. In headgear 404, however, caudal zygomatic stiffening element 464B is spaced from an intermediate occipital stiffening element 472C, with resultant lesser stiffness along that portion of occipital support 416 and resultant greater patient comfort. Similarly, parietal support 412 includes a posterior parietal stiffening element 466B that is likewise spaced from intermediate occipital stiffening element 472C.

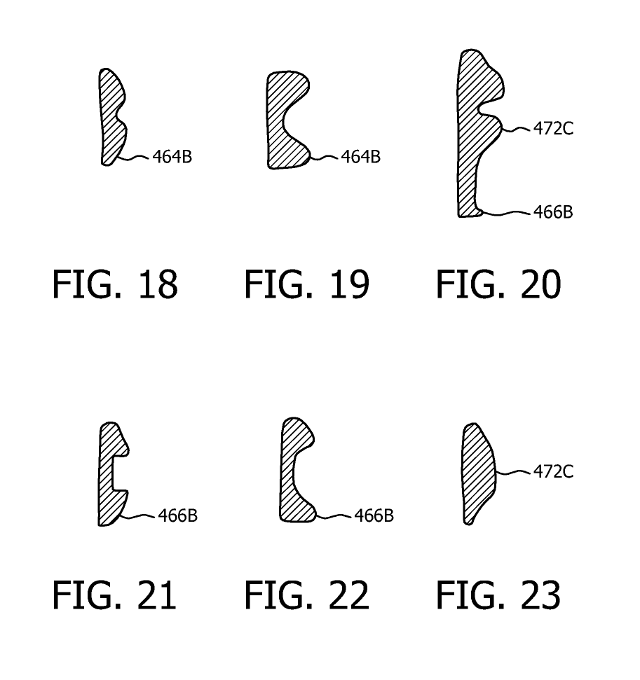

In contrast, FIG. 13 depicts posterior parietal stiffening element 366B of headgear 304 as extending along occipital support 316 to a point of engagement with intermediate occipital stiffening element 372C. Advantageously, therefore, by providing zygomatic brace 428 in headgear 404, it is possible to do away with cephalic and caudal occipital stiffening elements 372A and 372B, as were provided with headgear 304, which can increase patient comfort. Additionally or alternatively, by providing various combinations of elements, it is possible to provide a variety of models of headgear that are relatively more tolerated by individual patients, which is desirable. The various sectional views depicted in FIGS. 18-23 are provided merely as an example of how one such type of headgear can be configured, and is not intended to be limiting in any fashion whatsoever.

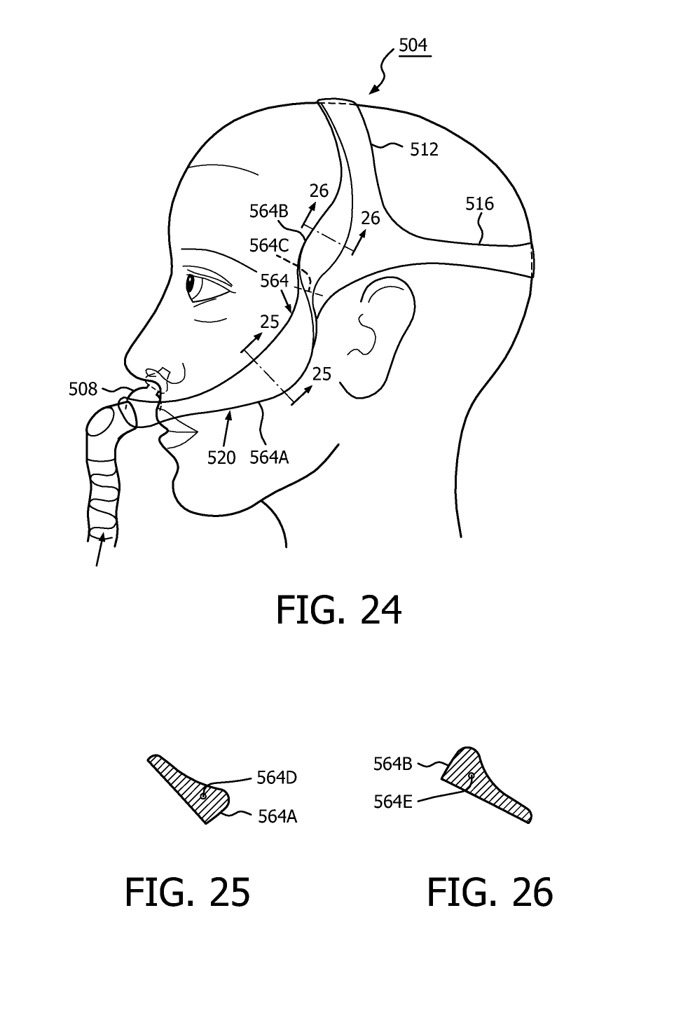

An improved headgear 504 in accordance with a seventh embodiment of the present invention is depicted generally in FIGS. 24-26. Headgear 504 maintains a nasal pillow interface 508 in fluid communication with the nostrils of a patient and includes a parietal support 512, an occipital support 516, and a zygomatic support 520 that are connected together. In headgear 504, zygomatic support 520 does not necessarily interact with the zygomatic bone of a patient in order to provide support. Headgear 504 includes a number of stiffening elements that maintain the position of zygomatic support 520 in relation to parietal support 512, which supports nasal pillow interface 508 in the nostrils of the patient.

More particularly, headgear 504 includes a stiffening element which can be characterized as a zygomatic/parietal stiffening element 564 that extends along at least a portion of zygomatic support 520 and along at least a portion of parietal support 512. Zygomatic/parietal stiffening element 564 is in the nature of a region of increased physical size in one or more directions transverse to the direction of elongation. FIG. 24 depicts at line 25-25 a section taken through a caudal segment 564A of zygomatic/parietal stiffening 564. FIG. 24 further depicts at line 26-26 a section through a cephalic segment 564B of zygomatic/parietal stiffening element 564B. Caudal segment 564A extends along zygomatic support 520, and cephalic segment 564B extends along a portion of zygomatic support 520 and along a portion of parietal support 512. Caudal segment 564A and cephalic segment 564B can be said to be separated by an inflection region 564C that is likewise depicted in FIG. 24.

As can be understood from FIG. 24, caudal segment 564A at the location of line 25-25 is roughly at its maximum dimension in a direction parallel to the plane of the page of FIG. 24, which is transverse to the direction of elongation of caudal segment 564A. Also at line 25-25 in FIG. 24, the thickness of caudal segment 564A as measured into the plane of the page of FIG. 24 varies in a fashion that can be seen in FIG. 25, and it is noted that such thickness is likewise a direction that is transverse to the direction of elongation of caudal segment 564A. The direction of elongation of zygomatic support 520 is generally along the direction extending between nasal pillow interface 508 and the connection with parietal support 512, and it can be understood from FIGS. 24 and 25 that in directions transverse to such direction of elongation, i.e., parallel with the plane of the page of FIG. 24 and into the plane of the page of FIG. 24, the transverse dimension of caudal segment 564A varies in order to achieve desirable degrees of stiffnesses in varying planes. The same can be said regarding cephalic segment 564B as is indicated along line 26-26 and in FIG. 26.

As can be seen from FIG. 25, the thickness of caudal segment 564A at line 25-25 is at a minimum at the edge adjacent the eye of the patient and is at a maximum at the edge adjacent the ear of the patient. In contrast, and as can be seen in FIG. 26, the thickness of cephalic segment 564B at line 26-26, is at a maximum at the edge adjacent the eye of the patient and is at a minimum at the edge adjacent the ear of the patient. Such reversal of cross-sectional shape occurs generally at inflection region 564C. Moreover, it can be seen that inflection region 564C has a smaller length in a direction parallel with the plane of the page of FIG. 24 than either the length of caudal segment 564A at line 25-25 parallel with the plane of the page of FIG. 24 and cephalic segment 564B at line 26-26 parallel with the plane of the page of FIG. 24.

By varying the thickness in a direction into the plane of the page of FIG. 24, as is particularly illustrated in FIGS. 25 and 26, the axis of bending of zygomatic/parietal stiffening element 564 can be controlled. For instance, when considering twisting of caudal segment 564A about the axis of elongation of caudal segment 564A, such twisting will occur along an axis running roughly through a centroid of the cross section as is indicated at a caudal centroid 564D in FIG. 25. That is, such twisting will occur about caudal centroid 564D rather than about a point midway along line 25-25. Likewise, twisting of cephalic segment 564B will occur along an axis that extends generally through a cephalic centroid 564E in FIG. 26 rather than about a point midway along line 26-26.

Strategic configuration of the dimensions and profiles of caudal and cephalic segments 564A and 564B and strategic positioning of inflection region 564C can provide desirable predetermined twisting and bending responses of zygomatic stiffening element 564 in order to enable headgear 504 to be mounted to various patients having various cranial and facial structures. As with certain other embodiments of the headgear set forth herein, headgear 504 is configured to have a fixed three-dimensional shape when in a free state. By carefully configuring zygomatic/parietal stiffening element 564, headgear 504 can be configured to have bending and twisting responses in various planes which enable headgear 504 to be comfortably fit on numerous patients having numerous cranial and facial structures while still comfortably and reliably maintaining nasal pillow interface 508 in fluid communication with the patient's nose.

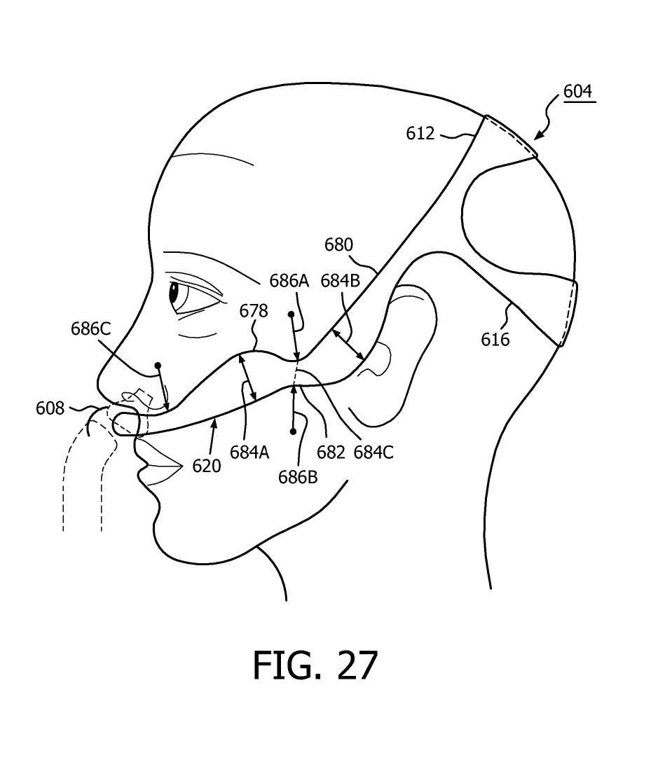

An improved headgear 604 in accordance with an eighth embodiment of the present invention is indicated generally in FIG. 27. Headgear 604 supports a nasal pillow interface 608 in fluid communication with the nostrils of a patient's nose and includes a parietal support 612, an occipital support 616, and a zygomatic support 620 that are connected together. While zygomatic support 620 in the side view presented in FIG. 27 shares some similarity in appearance with the side view of zygomatic support 520 in FIG. 24, it is noted that the thickness of zygomatic support 620 into the plane of the page of FIG. 27 does not vary as does that of headgear 504 as was indicated at FIGS. 25 and 26. Rather, zygomatic support 620 is configured to have a roughly constant thickness into the plane of the page of FIG. 27 along much of its length, and it employs other methodologies to support and maintain nasal pillow interface 608 in fluid communication with the nostrils of the patient.

More particularly, zygomatic support 620 includes an anterior support element 678, a posterior support element 680, and a hinge element 682 that is situated between anterior and posterior support elements 678 and 680. As will be set forth in greater detail below, hinge element 682 is configured to provide controlled deflection of anterior support element 678 with respect to posterior support element 680 in order to comfortably and reliably maintain nasal pillow interface 608 in fluid communication with the patient's nose. Anterior and posterior support elements 678 and 680 typically are of a generally fixed thickness into the plane of the page of FIG. 27, and they may be of substantially the same thickness (although this is not necessary). However, it is noted that hinge element 682 may have either a constant thickness or a varying thickness into the plane of the page of FIG. 27 if this is desired in order to provide a particular bending response of anterior support element 678 with respect to posterior support element 680. That is, hinge element 682 may be of the same thickness or a different thickness than anterior and posterior support elements 678 and 680, and the thickness may vary in a direction along the axis of elongation of zygomatic support 620 or a direction transverse thereto or a direction oblique thereto.

Anterior and posterior support elements 678 and 680 are not themselves necessarily intended to deflect to a meaningful extent within the plane of the page of FIG. 27, but rather hinge element 682 is intended to deflect in that plane when headgear 604 is positioned on the patient. This enables anterior support element 678 to be repositioned with respect to posterior support element 680 via deflection of hinge element 682. In this regard, anterior support element 678 may be configured to engage the zygomatic bone or zygomatic region of the patient in order to provide further support of nasal pillow interface 608.

FIG. 27 depicts anterior support element 678 having a transverse dimension 684A at one location therein and indicates posterior support element 680 having its own transverse dimension 684B at a location therein. Transverse dimensions 6874A and 684B are different than one another and are different than the transverse dimension of hinge element 682. Hinge element 682 itself is formed with a cephalic radius 686A and a caudal radius 686B which affect the deflection response of anterior support element 678 with respect to posterior support element 680 about hinge element 682. It is also noted that cephalic and caudal radii 686A and 686B are offset from one another along the longitudinal extent of zygomatic support 620. That is, the apex of cephalic radius 686A is not directly opposite the apex of caudal radius 686B, and rather the two apices of cephalic and caudal radii 686A and 686B are offset from one another longitudinally along zygomatic support 620B.

It is also noted that anterior support element 678 is formed with an anterior radius 686C in the vicinity of nasal pillow interface 608 which likewise affects the positioning of nasal pillow interface 608 within the nostrils of the patient. It is understood, however, that all of the various transverse dimensions and radii can be varied depending upon the needs of the particular application. For example, whereas transverse dimension 684A is greater than transverse dimension 684B, which is greater than transverse dimension 684C, it is understood that other relationships than those expressly depicted herein can be employed if desired to provide particularized bending responses about hinge element 682. Moreover, the various radii 686A, 686B, and 686C can additionally or alternatively be varied in magnitude and position to provide desired bending responses to achieve comfortable installation of headgear 604 on the patient while still maintaining support of nasal pillow interface 608 in the nostrils of the patient.

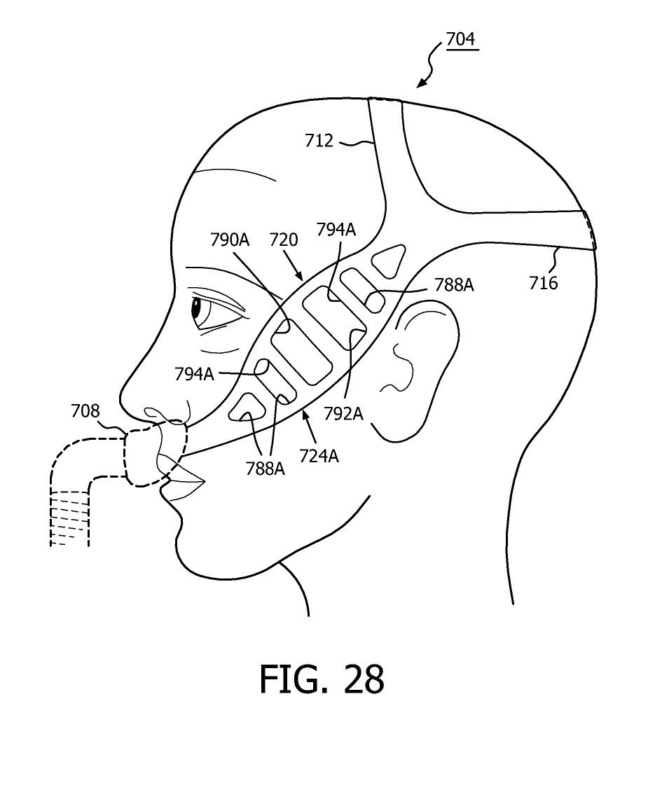

An improved headgear 704 in accordance with a ninth embodiment of the present invention is depicted in FIG. 28 installed on a patient. Headgear 704 supports a nasal pillow interface 708 in the nostrils of the patient and includes a parietal support 712, an occipital support 716, and a zygomatic support 720. While headgear 104, 204, 304, 404, 504, and 604, and variations thereof, have been indicated as being formed of a rigid but flexible silicone polymer or other material that has a specific three-dimensional shape when in a free state, it is noted that headgear 704 and its variations set forth below are instead formed of a flexible silicone rubber having a non-specific shape in a free state and are more flexible than rigid and are of an elastomeric arrangement.

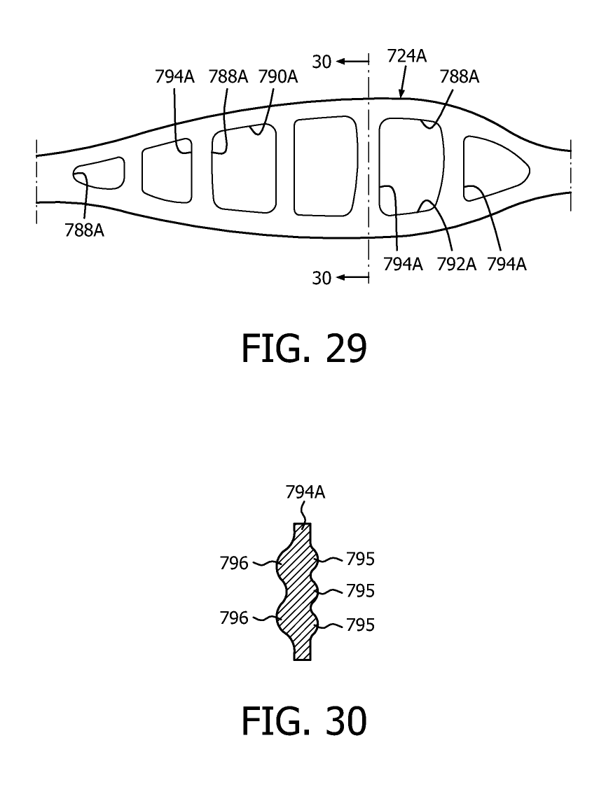

Zygomatic support 720 includes a zygomatic strap 724A that is more particularly shown in FIG. 29. Zygomatic strap 724A has a plurality of openings 788A formed therein that are of various shapes such as rectangular, triangular, etc., and are more particularly of a rounded rectangular or rounded triangular or other such shape as indicated generally in FIG. 29. Because of the various openings 788A formed in zygomatic strap 724A, zygomatic strap 724A can be said to include an elongated cephalic element 790A and an elongated caudal element 792A along with a plurality of transverse elements 794A that extend between cephalic and caudal elements 790A and 792A. As can be understood from FIG. 12, cephalic element 790A typically overlies the cephalic portion of the zygomatic bone, and caudal element 792A is situated caudal to the zygomatic bone, with at least some of transverse elements 794A overlying the crest of the zygomatic bone. Because the various openings 788A are formed in zygomatic strap 724A, the elastomeric cephalic, caudal, and transverse elements 790A, 792A, and 794A each serve as independent elastomeric strap segments that each independently deform and interact with the zygomatic bone to provide support to zygomatic strap 724A and to nasal pillow interface 708A.

One example of the cross sectional shape of transverse element 794A is provided along line 30-30 of FIG. 29 and is depicted in FIG. 30 wherein transverse element 794A includes a plurality of relatively smaller protrusions 795 that face generally toward the patient and a plurality of relatively larger protrusions 796 that face generally away from the patient. Such protrusions 795 are engaged with the skin of the patient and can be more comfortable than a relatively large flat strap engaged with a relatively large surface area of the skin. Also, protrusion 795 and 796 can be provided to desirably adjust the spring constant of transverse elements 794A as needed. Similar protrusions can be applied to other portions of zygomatic strap 724A as needed.

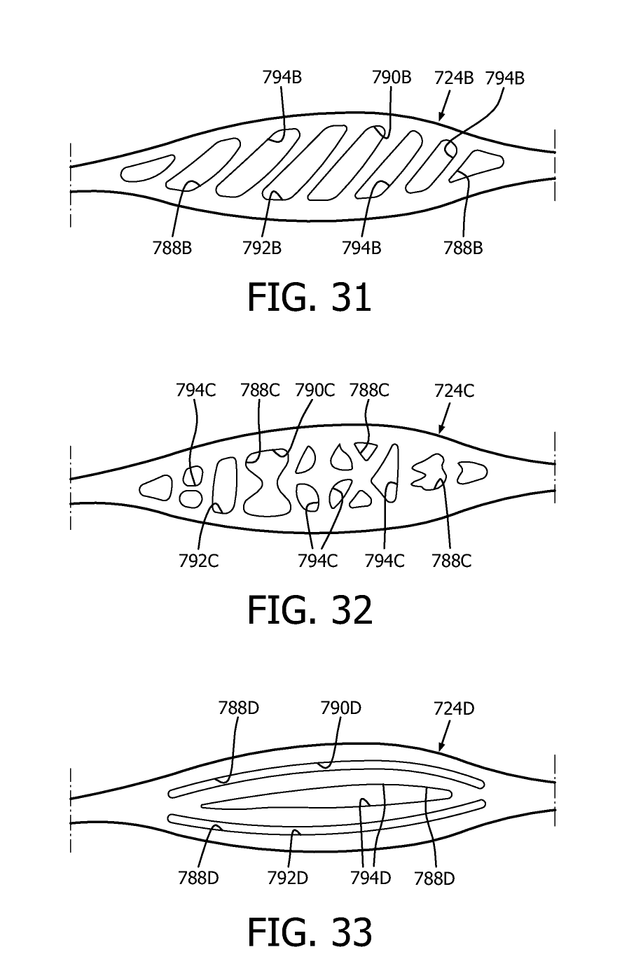

An alternative zygomatic strap 724B is depicted in FIG. 31 and can be employed in place of zygomatic strap 724A to result in a tenth embodiment of a headgear (not expressly depicted herein for simplicity of disclosure) in accordance with the present invention. Zygomatic strap 724B is formed with a plurality of openings 788B that are of a rounded parallelogram or rounded triangle or other such shape, with openings 788B resulting in zygomatic strap 724B having an elongated cephalic element 790B and an elongated caudal element 792B between which extend a plurality of transverse elements 794B. Zygomatic strap 724B is similar to zygomatic strap 724A except that the stretching and conforming of transverse elements 794B to a patient's zygomatic bone and potentially to the cranial structures will occur in a fashion slightly different from transverse elements 794A since transverse elements 794B will be oriented at a different angle with respect to the zygomatic bone. A headgear incorporating zygomatic strap 724B in accordance with the tenth embodiment may be more appropriate for a patient than headgear 704 of the ninth embodiment based, for example, on the particular shape of the zygomatic bones of the patient, or based upon other considerations.

FIG. 32 depicts another alternative zygomatic strap 724C that can be incorporated into headgear 704 in place of zygomatic strap 724A to form a headgear (not expressly depicted herein for simplicity of disclosure) in accordance with an eleventh embodiment of the present invention. Zygomatic strap 724C has a plurality of openings indicated generally at the numeral 788C and which are of various shapes that may be irregular, random, asymmetric, and the like depending upon the desired deflection characteristics of the resultant zygomatic strap 724C. Openings 788C result in zygomatic strap 724C having an elongated cephalic element 790C, an elongated caudal element 792C, and a plurality of transverse elements indicated generally at the numeral 794C which extend between and among cephalic, caudal, and other transverse elements 790C, 792C, and 794C. That is, it can be seen that certain transverse elements extend between cephalic and caudal elements 790C and 792C. Some transverse elements 794C can be seen to extend between a pair of other transverse elements 794C. Still other transverse elements 794C can be seen to extend between another transverse element 794C and either cephalic element 790C or caudal element 792C. It thus can be seen that an unlimited variety of shapes and arrangements of openings 788C can be provided in order to impart to zygomatic strap 724C any of an unlimited variety of elastic response profiles that can comfortably and reliably support nasal pillow interface 708 on a patient.

Another alternative zygomatic strap 724D that can be used in place of zygomatic strap 724A to form a headgear (not expressly depicted herein for simplicity of disclosure) in accordance a twelfth embodiment of the present invention is indicated generally in FIG. 33. Zygomatic strap 724D has a plurality of elongated openings 788D that extend generally along the axis of elongation of zygomatic strap 724D. The result of the formation of openings 788D is to produce in zygomatic strap 724D an elongated cephalic element 790D, an elongated caudal element 792D, and a pair of elongated longitudinal elements 794D that are situated intermediate cephalic and caudal elements 790D and 792D. Because openings 788D separate from one another the cephalic, caudal, and longitudinal elements 790D, 792D, and 794D, each operates as an independent elastomeric element that can engage the face of the patient in the vicinity of the zygomatic bone. In one embodiment, zygomatic strap 724D might be positioned such that the crest of the zygomatic bone extends through central opening 788D such that longitudinal elements 794D are situated cephalic and caudal to the crest of the zygomatic bone. Other installations are possible. Likewise, it is understood that other arrangements of such elongated openings 788D can be employed to generate other deflection properties in the resultant zygomatic strap 724D.

It is understood that the various features of zygomatic straps 724A, 724B, 724C, and 724D can be combined with one another in any of a variety of fashions and can be combined with other elements in order to provide different combinations of elements that are still within the scope of the present disclosure. Moreover, various elements from any of zygomatic straps 724A, 724B, 724C, and 724D can be employed in conjunction with any of the elements of the foregoing headgear 4, 104, 204, 304, 404, 504, and 604. The various embodiments and possible combinations thereof advantageously enable therapeutic retention of a nasal pillow interface on a patient while provided enhanced comfort to the patient.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A headgear structured for use in securing a mask to the head of a patient, the headgear comprising:
a parietal support, at least a portion of which is structured to extend across a parietal region of the head;
an occipital support, at least a portion of which is structured to extend across an occipital region of the head; and
a zygomatic support, at least a portion of which is structured to extend across a zygomatic region of the head, wherein the parietal, occipital, and zygomatic supports being connected together, and wherein the zygomatic support comprises a pair of zygomatic braces that are each structured to engage the face of the patient and to support the mask in fluid communication with the patient, the zygomatic braces each comprising at least one of:
a cephalic element structured to engage the face of the patient in a region cephalic to the crest of the zygomatic bone, and
a caudal element being structured to engage the face of the patient in a region caudal to the crest of the zygomatic bone.

2. The headgear of claim 1, wherein the zygomatic braces each comprise both of the cephalic element and the caudal element.

3. The headgear of claim 2, wherein the zygomatic braces each further comprise a linking element in the form of a region of reduced thickness having a greater deformability than the cephalic and caudal elements, the linking element extending between at least a portion of the cephalic element and at least a portion of the caudal element.

4. The headgear of claim 3, wherein the zygomatic braces each further comprise an intermediate element connected with the linking element, the intermediate element being structured to engage the face of the patient in a region at the crest of the zygomatic bone.

5. The headgear of claim 4, wherein the linking element extends about substantially the entirety of the intermediate element to isolate substantially the entirety of the intermediate element from the cephalic and caudal elements.

6. The headgear of claim 4, wherein at least a portion of the intermediate element extends between at least a portion of the cephalic element and at least a portion of the caudal element.

7. The headgear of claim 6, wherein the region of reduced thickness comprises a region of reduced thickness disposed anterior to the intermediate element and another region of reduced thickness disposed posterior anterior to the intermediate element.

8. The headgear of claim 1, wherein the pair of zygomatic braces are each in the form of a cup that is structured to engage the zygomatic region of the face and to support the mask in fluid communication with the patient.

9. The headgear of claim 1, wherein the zygomatic support further comprises a zygomatic strap, at least a portion of the zygomatic strap being disposed posterior to each zygomatic brace and being of a thickness in a plane toward the patient and having a transverse dimension along an axis transverse to the thickness that is a plurality of times greater than the thickness to permit bending of the zygomatic strap about the axis and to resist bending of the zygomatic strap about another axis transverse to the axis.

10. The headgear of claim 9 wherein the at least portion of the zygomatic strap comprises a pair of curve elements structured to position the zygomatic braces in proximity to the zygomatic regions of the face of the patient.

11. A headgear apparatus comprising:
(a) a nasal interface mask structured to be in fluid communication with the head of a patient; and
(b) a headgear structured to support the nasal interface mask in fluid communication with the head of the patient, the headgear comprising:
(1) a parietal support, at least a portion of which is structured to extend across a parietal region of the head;
(2) an occipital support, at least a portion of which is structured to extend across an occipital region of the head;
(3) a zygomatic support, at least a portion of which is structured to extend across a zygomatic region of the head, wherein the parietal, occipital, and zygomatic supports being connected together, and wherein the zygomatic support comprises a pair of zygomatic braces that are each structured to engage the face of the patient and to support the mask in fluid communication with the patient, the zygomatic braces each comprising at least one of:
(i) a cephalic element structured to engage the face of the patient in a region cephalic to the crest of the zygomatic bone, and
(ii) a caudal element being structured to engage the face of the patient in a region caudal to the crest of the zygomatic bone.

12-14. (canceled)

15. The headgear apparatus of claim 11 wherein the zygomatic braces each comprise both of the cephalic element and the caudal element;
wherein the zygomatic braces each further comprise a linking element in the form of a region of reduced thickness, the linking element extending between at least a portion of the cephalic element and at least a portion of the caudal element;
wherein the zygomatic braces each further comprise an intermediate element connected with the linking element, the intermediate element being structured to engage the face of the patient in a region at the crest of the zygomatic bone; and
wherein the linking element extends about substantially the entirety of the intermediate element to isolate substantially the entirety of the intermediate element from the cephalic and caudal elements.

16. (canceled)

17. The headgear apparatus of claim 11:
wherein the zygomatic braces each comprise both of the cephalic element and the caudal element;
wherein the zygomatic braces each further comprise a linking element in the form of a region of reduced thickness, the linking element extending between at least a portion of the cephalic element and at least a portion of the caudal element;
wherein the zygomatic braces each further comprise an intermediate element connected with the linking element, the intermediate element being structured to engage the face of the patient in a region at the crest of the zygomatic bone;
wherein at least a portion of the intermediate element extends between at least a portion of the cephalic element and at least a portion of the caudal element; and
wherein the region of reduced thickness comprises a region of reduced thickness disposed anterior to the intermediate element and another region of reduced thickness disposed posterior anterior to the intermediate element.

18. The headgear apparatus of claim 11, wherein the pair of zygomatic braces are each in the form of a cup that is structured to engage the zygomatic region of the face and to support the mask in fluid communication with the patient.

19. (canceled)

20. The headgear apparatus of claim 11:
wherein the zygomatic support further comprises a zygomatic strap, at least a portion of the zygomatic strap being disposed posterior to each zygomatic brace and being of a thickness in a plane toward the patient and having a transverse dimension along an axis transverse to the thickness that is a plurality of times greater than the thickness to permit bending of the zygomatic strap about the axis and to resist bending of the zygomatic strap about another axis transverse to the axis; and
wherein the at least portion of the zygomatic strap comprises a pair of curve elements structured to position the zygomatic braces in proximity to the zygomatic regions of the face of the patient.
